(19) United States
(12) Patent Application Publication
(10) Pub. No.: US 2005/0019752 A1
(43) Pub. Date: Jan. 27, 2005

(54) Novel chimeric rev, tat, and nef antigens

(76) Inventors: Genoveffa Franchini, Washington, DC (US); Zdenek Hel, Birmingham, AL (US); James Tartaglia, Aurora (CA)

Correspondence Address: Schwegman Lundberg;Woessner & Kluth, PO Box 2938, Minneaplis, MN, 55402, US

(21) Appl. No.: 10/495,532

(22) PCT Filed: Nov. 15, 2002
(86) PCT No.: PCT/US02/36805

Related U.S. Application Data
(60) Provisional application No. 60/332,433, filed on Nov. 16, 2001.

(51) Int. Cl. C12Q 1/70; C07H 21/04; C12N 15/86; C07K 14/005

(52) U.S. Cl. 435/5; 435/69.3; 435/456; 435/325; 435/235.1; 530/350; 536/23.72

(57) ABSTRACT

This invention provides novel HIV antigens comprising chimeric rev, tat, and nef for use in inducing an immune response. The novel antigens can be used as vaccines to prevent and/or attenuate HIV infection.

### CROSS REFERENCE TO RELATED APPLICATIONS

This application claims benefit of priority of U.S. Provisional Application No. 60/332,433, filed Nov. 16, 2001, which is herein incorporated by reference.

### FIELD OF THE INVENTION

This invention relates to improved methods of inducing an immune response for the prevention or treatment of human immunodeficiency virus (HIV) infection by using a novel chimeric antigen that comprises genetically modified and re-assorted rev, tat, and nef genes.

### BACKGROUND OF THE INVENTION

An effective vaccine for treatment or prevention of HIV infection desirably induces a high frequency of CTL responses to multiple epitopes, as virus specific CD8+ T-cell responses have been associated with viremia containment in HIV or SIV infected humans or macaques, respectively (Allen, T. M. et al., Nature, 407:386-390 (2000); Borrow et al., J. Virol., 68:6103-6110 (1994); Borrow et al., Nat.Med., 3:205-211 (1997); Goulder et al., AIDS, 13 Suppl A:S121-S136 (1999); Koup et al., J. Virol., 68:4650-4655 (1994); Letvin et al., Science, 280:1875-1880 (1998); McMichael et al., Nature, 410:980-987 (2001); Rinaldo et al., J. Virol., 69:5838-5842 (1995); Rowland-Jones et al., Curr Opin Immunol, 7:448-455 (1995); Schmitz et al., Science, 283:857-860 (1999)). Most of the vaccines developed against HIV-1, SIV-1, or chimeric SHIV viruses have included the viral structural env and gag genes, and, in some cases, the protease gene. These vaccines, however, have not prevented viral infection, although, in some cases, a milder disease course was observed (Amara et al., Science, 292:69-74 (2001); Benson et al., J. Virol., 72:4170-4182 (1998); Hanke et al., J. Virol., 73:7524-7532 (1999); Hirsch et al., J. Virol., 70:3741-3752 (1996); Kent et al., J. Virol., 72:10180-10188 (1998); Ourmanov et al., J. Virol., 74:2740-2751 (2000); Robinson et al., Nat.Med., 5:526-534 (1999); Seth et al., J. Virol., 74:2502-2509 (2000)).

The response against Rev, Tat, and Nef proteins, which are expressed early in the virus life cycle (Cullen, FASEB J, 5:2361-2368 (1991); Robert-Guroffet al., J. Virol., 64:3391-3398 (1990)), may be particularly important in viral containment because their recognition may occur before Nef down-modulates MHC class I molecules on the surface of the infected cells (Collins et al., Nature, 391:397-401 (1998)) prior to assembly of viral particles. This would therefore provide a window of opportunity to the immune system to eliminate the infected cell before virus is released. In addition, as Nef protects HIV-infected cells from apoptosis (Antoni et al., J Virol, 69:2384-2392 (1995); Cullen, FASEB J, 5:2361-2368 (1991); Robert-Guroff et al., J. Virol., 64:3391-3398 (1990); Yoon et al., AIDS Res Hum Retroviruses, 17:99-104 (2001)), a prompt recognition of Nef-expressing cells by cytotoxic T-lymphocytes (CTL) may increase the chances of eliminating virus-infected cells. The functional domains of Tat and Rev proteins are relatively well conserved among different HIV-1 clades (Meyers et al., B. Korber (ed.), Human retroviruses and AIDS. Theoretical Biology and Biophysics, Los Alamos National Laboratory, Los Alamos, p. 55-56 (1995)) and are targets of CTL's in HIV-1-infected individuals, particularly in the acute phase of infection (Addo et al., Proc Natl Acad Sci USA, 98:1781-1786 (2001); Novitsky et al., J Virol, 75:9210-9228 (2001); van Baalen et al., J Gen Virol, 78 (Pt 8):1913-1918 (1997)). Responses to these viral proteins have been identified also in long term non-progressor individuals who contain viremia in an absence of antiretroviral therapy (Addo et al., Proc Natl Acad Sci USA, 98:1781-1786 (2001); Froebel et al., AIDS Res Hum Retroviruses, 10 Suppl 2:S83-S88 (1994); Novitsky et al., J Virol, 75:9210-9228 (2001)). Induction of immune responses to Tat and Rev may therefore also be important in protection from or potentiation of the immune response to HIV.

In particular, CTL responses directed against functionally important domains of these regulatory proteins may reduce the emergence of viral immune escape, as mutation at these sites may reduce viral fitness (Nietfield et al., J Immunol, 154:2189-2197 (1995)). Indeed, in some patients, the relative frequency of CTL recognition has been reported to be higher for Rev, Tat, and Nef than reverse transcriptase, Env gp41, or gp120 epitopes (Addo et al., Proc Natl Acad Sci USA, 98:1781-1786(2001)). The relatively high frequency of anti-Tat response observed in some studies could be explained by its availability in extracellular compartments and uptake from cells, as implied by in vitro studies (Cafaro et al., Nat. Med., 5:643-650 (1999); Kim et al., J Immunol, 159:1666-1668 (1997)).

The ability of early regulatory protein-specific CTL to exert immunological pressure has been demonstrated in the macaque model in which selection for viral immune escape variants in Tat (Allen, T. M. et al., Nature, 407:386-390 (2000)) and Nef (Evans et al., Nat. Med., 5:1270-1276 (1999); Mortara et al., Virology, 278:551-561 (2000)) occurs frequently during primary SIVmac infection. In this model, as well as in humans infected with HIV-1, the presence of humoral and cellular immune responses against Tat (Froebel et al., AIDS Res Hum Retroviruses, 10 Suppl 2:S83-S88 (1994); Re et al., J Acquir Immune Defic Syndr Hum Retrovirol, 10:408-416 (1995); Rodman et al., Proc Natl Acad Sci USA, 90:7719-7723 (1993); Venet et al., J Immunol, 148:2899-2908 (1992); Zagury et al., J Hum Virol, 1:282-292 (1998)) and CTL responses against Rev (van Baalen et al., J Gen Virol, 78 (Pt 8):1913-1918 (1997); van Baalen et al., J Virol, 72:6851-6857 (1998)) and Nef (Sriwanthana et al., AIDS Res Hum Retroviruses, 17:719-734 (2001)) have been demonstrated to inversely correlate with disease progression.

The potential benefits of the inclusion of regulatory proteins as part of vaccines against immunodeficiency viruses has been investigated. In macaques, immunization with biologically active Tat protein (Cafaro et al., Nat. Med., 5:643-650 (1999)), Tat toxoid (Pauza et al., Proc. Natl. Acad. Sci. USA, 97:3515-3519 (2000)), Tat-encoding DNA vaccine (Cafaro et al., Vaccine, 19:2862-2877 (2001)), or recombinant vaccinia vectors expressing Tat and Rev proteins (Osterhaus et al., Vaccine, 17:2713-2714 (1999)) did not protect from infection and have demonstrated various levels of attenuation of virus replication and disease progression, depending on the animal model used.

A protective effect of Nef-specific CTLs has been established in a study in macaques, where an inverse correlation was found between the vaccine-induced Nef-specific CTL precursor frequency and virus load measured after challenge (Gallimore et al., Nat. Med., 1:1167-1173 (1995)). However, other studies have shown only limited immune responses elicited by vaccination with early regulatory genes and no protection against viral challenge (Calarota et al., J. Immunol., 163:2330-2338 (1999); Nilsson et al., Vaccine, 19:3526-3536 (2001); Putkonen et al., Virology, 250:293-301 (1998)). In most studies, except two (Pauza et al., Proc. Natl. Acad. Sci. USA, 97:3515-3519 (2000)), both Tat and Nef proteins have been used without prior modification.

Expression of HIV and SIVmac genes, including Rev, Tat, and Nef, in mammalian cells has proven to be difficult because of a highly distinct codon bias for adenine and thymidine at the third codon position (Andre et al., J Virol, 72:1497-1503 (1998); Haas et al., Curr Biol, 6:315-324 (1996)), which limits their translation efficiency. Furthermore, several in vitro studies indicate that both Nef and Tat may have negative effects on the host immune response. For example, Nef down-modulates MHC-I, CD4, and CD28 (Carl et al., J Virol, 75:3657-3665 (2001); Collins et al., Nature, 391:397-401 (1998); Swigut et al., EMBO J, 20:1593-1604 (2001)), induces T-cell hyporesponsiveness (Collette et al., J Biol Chem, 271:6333-6341 (1996); Collette et al., Eur J Immunol, 26:1788-1793 (1996)), and up-regulates Fas ligand on the surface of infected cells (Xu et al., J Exp Med, 186:7-16 (1997)). Wild type Tat also exerts immunosuppressive functions, through inhibition of both antigen-driven and nonspecific T-cell proliferation (Collette et al., J Biol Chem, 271:6333-6341 (1996); Collette et al., Eur J Immunol, 26:1788-1793 (1996); Viscidi et al., Science, 246:1606-1608 (1989); Wrenger et al., J Biol Chem, 272:30283-30288 (1997)), induction of T-cell apoptosis (Goldstein, Nat Med, 2:960-964 (1996)), inhibition of phagocytosis by accessory cells (Zocchi et al., AIDS, 11:1227-1235 (1997)), inhibition of IL-2 secretion (Poggi et al., J Biol Chem, 273:7205-7209 (1998)), and down-regulation of MHC class II complexes (Kanazawa et al., Immunity, 12:61-70 (2000)).

Other deleterious effects of these regulatory proteins have also been reported. Tat transactivates multiple cellular genes, including a number of cytokines, intercellular adhesion molecules, and chemokines (Rubartelli et al., Immunol. Today, 19:543-545 (1998)) and induces angiogenesis, possibly contributing to the development of AIDS-associated tumors (Albini et al., Nat Med, 2:1371-1375 (1996). Tat and Rev proteins induce defects in neuronal differentiation and neuronal death (Mabrouk et al., FEBS Lett, 289:13-17 (1991); Nath et al., J Virol, 70:1475-1480 (1996) and Nef promotes neoplastic transformation of immortalized neural cells in vitro (Kramer-Hammerle et al., AIDS Res Hum Retroviruses, 17:597-602 (2001)).

Thus, there is a need to develop safe and efficacious vaccines that include regulatory HIV proteins. The current invention addresses this need and provides a novel chimeric gene comprising genetically modified and reassorted rev, tat, and nef genes, e.g. retanef. Such proteins, which are typically expressed in the cytoplasm, are safe and immunogenic vaccines for the prevention and treatment of HIV infection.

### SUMMARY OF THE INVENTION

The invention provides expression vectors for the prevention or treatment of HIV infection. In particular, the invention provides an expression vector comprising a nucleic acid encoding a chimeric rev, tat, and nef polypeptide. The expression vector is often a viral vector, such as an attenuated pox virus vector, e.g., NYVAC, ALVAC, MVA, or fowlpox. Other viral vectors can also be used, these include adenovirus vectors, adeno-associated virus vector, or Venezuelan equine encephalomyelitis virus vectors.

The chimeric polypeptide expressed by the vector typically comprises functional domains of tat and nef that are disrupted. Furthemore, the chimeric polypeptide expressed by the vector can lack a Rev nuclear localization signal, a Rev RNA-binding domain, and a Tat RNA-binding domain; and/or can lack an N-terminal Nef myristylation signal. In one embodiment, the expression vector comprises a retanef gene, which encodes the polypeptide Retanef. Often, the retanef gene is expressed using a NYVAC or ALVAC vector.

In another aspect, the invention provides a method of inducing an immune response comprising administering a first expression vector comprising a nucleic acid sequence encoding a chimeric rev, tat, and nef polypeptide, wherein the expression vector enters the cells of the recipient and intracellularly produces rev, tat, and nef-specific peptides that are presented on the cell's MHC class I molecules in an amount sufficient to stimulate a CD8⁺ response. The method often employs an expression vector, e.g., a viral expression vector, encoding retanef.

The method often further comprise administering a second expression vector comprising a nucleic acid sequence encoding a chimeric rev, tat, and nef polypeptide, e.g., retanef, wherein the second expression vector is administered as naked DNA. In some embodiments, the naked DNA expressing the chimeric rev, tat, and nef polypeptide is administered prior to a viral vector that expressed a chimeric rev, tat, and nef polypeptide. In other embodiments, the method comprises additional steps of administering one or more expression vectors, e.g., NYVAC and/or naked DNA, encoding HIV structural polypeptides such as one or more epitopes from the gag, pol, and env genes.

### BRIEF DESCRIPTION OF THE FIGURES

FIG. 1 shows a schematic representation of the chimeric Retanef protein. The exons of Tat, Nef, and Rev are arranged in the configuration depicted in the figure. C=carboxy terminus. N=amino terminus. The “number” refers to the amino acid number of the proteins according to the Los Alamos database for SIV.

FIG. 2 shows the nucleotide and amino acid sequence of the chimeric retanef gene and the amino acid sequence of the Retanef protein.

FIG. 3 shows tat-specific T-cell proliferation in macaques inoculated with DNA/NYVACretanef alone and macaques inoculated with DNA/NYVACretanef and DNA/NYVACgag-pol-env (lower panel).

FIG. 4 shows nef-specific T-cell proliferation in macaques inoculated with DNA/NYVACretanef alone and macaques inoculated with DNA/NYVACretanef and DNA/NYVACgag-pol-env (lower panel).

FIG. 5 shows Tat28 tetramer staining in macaques inoculated with DNA/NYVACretanef alone and macaques inoculated with DNA/NYVACretanef and DNA/NYVACgag-pol-env (lower panel).

FIG. 6 shows the level of SIV in the blood of animals vaccinated with the constructs as indicated for each group.

FIG. 7 shows the statistical significance of the data shown in FIG. 6.

Definitions

“Attenuated recombinant virus” refers to a virus that has been genetically altered by modern molecular biological methods, e.g. restriction endonuclease and ligase treatment, and rendered less virulent than wild type, typically by deletion of specific genes or by serial passage in a non-natural host cell line or at cold temperatures.

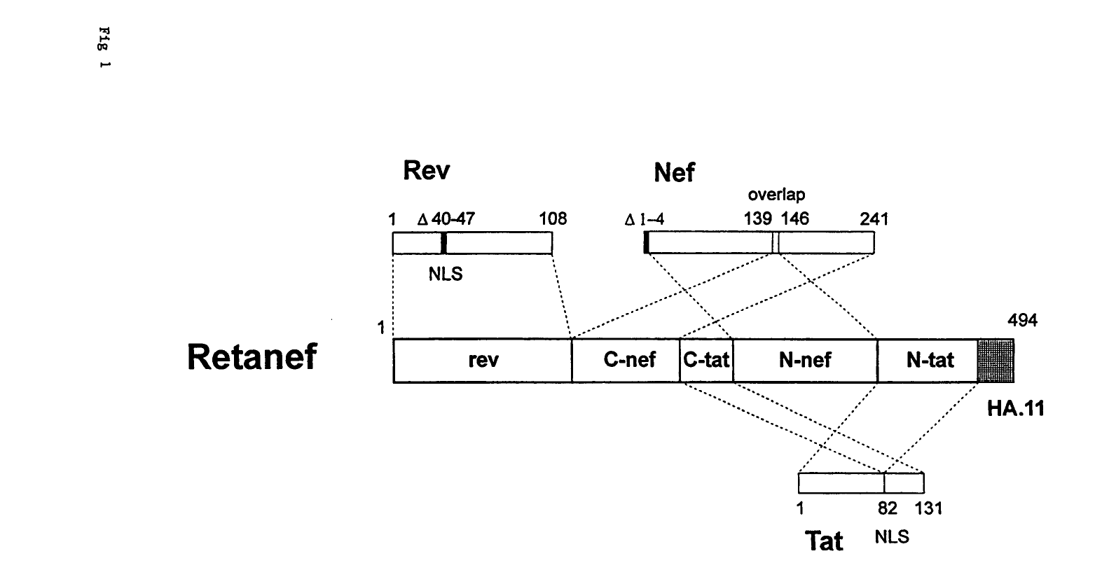

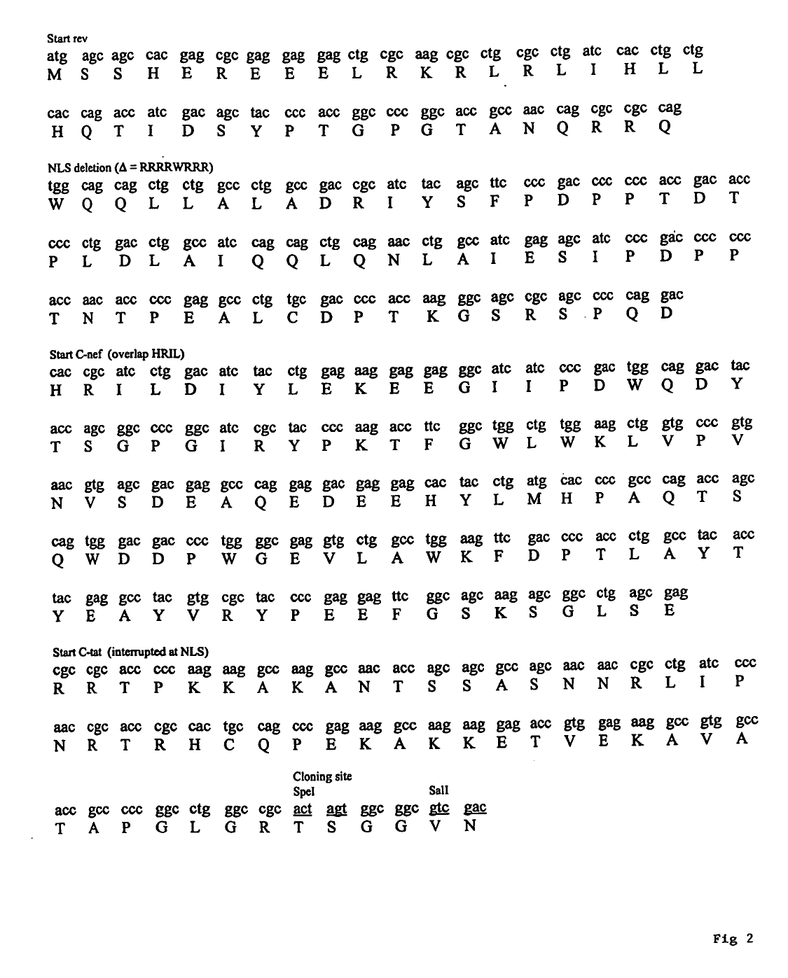

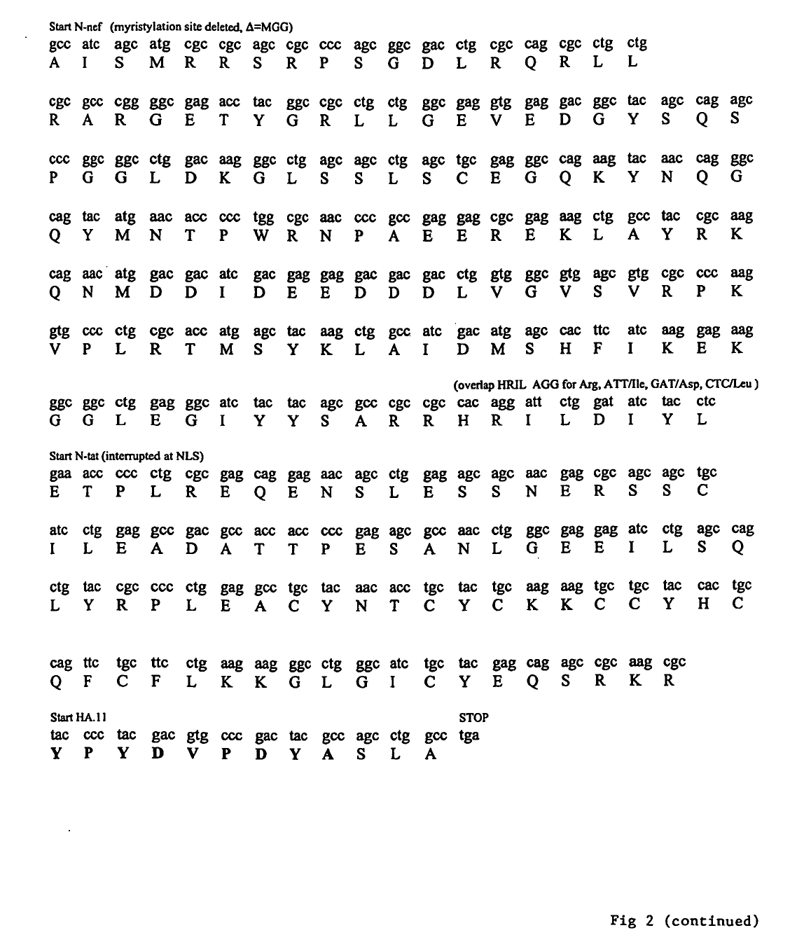

A “chimeric rev, tat, and nef polypeptide” refers to a fusion protein, i.e., the sequences are covalently linked, comprising rev, tat, and nef polypeptide sequences, or subsequences (also referred to as “fragments” or “domains”), thereof. Rev, tat, and nef polypeptide sequences can be in any order in the chimeric molecules. Fragments of the polypeptides comprising the chimeric constructs can also be interspersed, e.g., subsequences of rev can be interspersed with subsequences of tat or nef. “Retanef” refers to the construct shown in FIG. 1. The nucleic acid and amino acid sequences of the Retanef construct in FIG. 1 is provided in FIG. 2. The term “Retanef” includes conservatively modified variants that induce at least 70%, preferably, 80%, 85%, 90%, or 95% of the immune response induced by the Retanef polypeptide having the amino acid sequence set forth in FIG. 2. The characterization of such proteins is described in the sections herein describing chimeric rev-tat-nef polypeptides and methods of analyzing the immune response.

“Efficient CD8⁺ response” is referred to as the ability of cytotoxic CD8⁺ T-cells to recognize and kill cells expressing foreign peptides in the context of a major histocompatibility complex (MHC) class I molecule.

“Nonstructural viral proteins” are those proteins that are needed for viral production but are not found as components of the viral particle. They include DNA binding proteins and various enzymes that are encoded by viral genes. The term “proteins” includes both the intact proteins and fragments of the proteins or peptides which are recognized by the immune cell as epitopes of the native protein.

A “nucleic acid vaccine” or “naked DNA vaccine” refers to a vaccine that includes one or more expression vectors that encodes B-cell and/or T-cell epitopes and provides an immunoprotective response in the person being vaccinated. As used herein, the term does not include a viral vaccine, i.e., a vaccine in which the nucleic acid is within a viral capsid. An “expression vector” refers to any expression vector, e.g., viral or plasmid.

“Nucleic acid-based vaccines” can include both naked DNA and vectored DNA within a viral capsid where the nucleic acid encodes B-cell and T-cell epitopes and provides an immunoprotective response in the person being vaccinated.

The term “reassorted” as used herein refers to splitting at least one of rev, tat, or nef proteins into segments that are separated in the chimeric polypeptide such that the activity of the polypeptide or a domain of the polypeptide is disrupted.

“Pox viruses” are large, enveloped viruses with double-stranded DNA that is covalently closed at the ends. Pox viruses replicate entirely in the cytoplasm, establishing discrete centers of viral synthesis. Their use as vaccines has been known since the early 1980's (see, e.g. Panicali, D. et al. “Construction of live vaccines by using genetically engineered pox viruses: biological activity of recombinant vaccinia virus expressing influenza virus hemagglutinin”, Proc. Natl. Acad. Sci. USA 80:5364-5368, 1983).

“Potentiating” or “enhancing” an immune response means increasing the magnitude and/or the breadth of the immune response, i.e., the number of cells induced by a particular epitope may be increased and/or the numbers of epitopes that are recognized may be increased (“breadth”). A 5-fold, often 10-fold or greater, enhancement in both CD8⁺ and CD4⁺ T-cell responses is obtained with administration of a combination of nucleic acid/recombinant virus vaccines compared to administration of either vaccine alone.

A “retrovirus” is a virus containing an RNA genome and an enzyme, reverse transcriptase, which is an RNA-dependent DNA polymerase that uses an RNA molecule as a template for the synthesis of a complementary DNA strand. The DNA form of a retrovirus commonly integrates into the host-cell chromosomes and remains part of the host cell genome for the rest of the cell's life.

“Viral load” is the amount of virus present in the blood of a patient. Viral load is also referred to as viral titer or viremia. Viral load can be measured in variety of standard ways. In preferred embodiments, the DNA/recombinant virus prime boost protocol of the invention controls viremia and leads to a greater reduction in viral load than that obtained when either vaccine is used alone.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a BLAST or BLAST 2.0 sequence comparison algorithms with default parameters described below, or by manual alignment and visual inspection. Such sequences are then said to be “substantially identical.” This definition also refers to the complement of a test sequence. Preferably, the identity exists over a region that is at least about 20 amino acids or nucleotides in length, or more preferably over a region that is 25, 35, or 50-100 amino acids or nucleotides in length.

### DETAILED DESCRIPTION

Introduction

The human immunodeficiency virus (HIV), regulatory proteins Rev, Tat, and Nef are expressed early after infection and represent attractive targets to be included in a vaccine for AIDS. However, the low level expression of these proteins in mammalian cells and the potential immunosuppressive activity of Tat and Nef, as such, represent a limitation to their inclusion in preventive or therapeutic vaccines. To circumvent these issues, the current invention provides novel polynucleotides and polypeptides comprising tat, rev, and nef chimeric molecules. The chimeric molecules of the invention comprises genetically modified and re-assorted rev, tat, and nef genes, e.g., retanef, which encodes an immunogenic protein of approximately 55 kDa. Retanef and other chimeric polypeptides of the invention can be provided as a vaccine for the prevention or attentuation of HIV infection.

Typically, the vaccine is a nucleic acid-based vaccine, often a plasmid eucaryotic expression vector or a recombinant viral vector, e.g., an attenuated pox viruses vector. The vaccine can be administered to individuals at risk for infection or individuals who may already be infected.

Vaccines of Use in this Invention

Vaccines useful for the induction of CD8⁺ T-cell responses comprise nucleic acid-based vaccines (preferably delivered as a DNA-based vaccine) including naked DNA and viral vectors, e.g., recombinant pox virus vaccines, that provide for the intracellular production of viral-specific peptide epitopes that are presented on MHC Class I molecules and subsequently induce an immunoprotective cytotoxic T lymphocyte (CTL) response.

The invention typically contemplates single or multiple administrations of the nucleic acid vaccine in combination with one or more administrations of the recombinant virus vaccine. This vaccination regimen may be complemented with administration of recombinant protein vaccines, or may be used with additional vaccine vehicles. Preferably, administration of the nucleic acid vaccine precedes administration of the recombinant virus vaccine.

In preferred embodiments, the DNA/recombinant virus prime boost protocol controls viremia and reduces viral load as well as potentiating a CD8⁺ response.

Chimeric rev-tat-nef

Chimeric nucleic acids of the invention comprise the non-structural genes rev, tat, and nef of HIV. The polypeptide encoded by the nucleic acid is thus a fusion polypeptide comprising regions of rev, tat, and nef, i.e., the regions of rev, tat, and nef are covalently bonded to one another.

The rev, tat, and nef sequences can be from any HIV sequence, including both HIV-1 and HIV-2. Rev, tat, and nef nucleic acid and protein sequences for use in generating the chimeric molecules of the invention are known, e.g., the Los Alamos database. The HIV-1 and HIV-2 genomes, and the DNA sequences of HIV-1 and HIV-2, and respective strains are also described in the publication, HIV Sequence Compendium 2000, Kuiken et al, Eds. Theoretical Biology and Biophysics Group, Los Alamos National Laboratory, Los Almos, N. Mex. Any rev, tat, or nef sequence can be used to construct the vaccines of the invention.

Construction of the vaccine constructs uses routine techniques in the field of recombinant genetics. Basic texts disclosing the general methods of use in this invention include Sambrook and Russell, Molecular Cloning, A Laboratory Manual (3rd ed. 2001, Cold Spring Harbor Laboratory Press); Kriegler, Gene Transfer and Expression: A Laboratory Manual (1990); and Current Protocols in Molecular Biology (Ausubel et al., eds., 1994)).

The rev, tat, and nef sequences included in the chimeric proteins of the invention are typically disrupted and/or altered to inactivate specific functions of the individual proteins. The specific functions can be inactivated using a variety of approaches, for example, by shuffling the coding regions of the nucleic acid sequences encoding the proteins; by deleting specific regions of the individual proteins, e.g., one or more amino acid residues in a functional domain of the protein; or by mutagenizing specific sequences to prevent function. Such changes are typically accomplished so as to prevent disruption of known T-cell, or B-cell, epitopes. Accordingly, these chimeric constructs retain immunogenicity.

There are many ways of generating alterations in a given nucleic acid sequence. Many methods, for example used amplifcation, e.g., PCR amplifcation strategies, to amplify sequences containing the desired changes. Alternatively, a nucleic acid of the invention can be chemically synthesized.

For chemical synthesis, the peptides can be synthesized in solution or on a solid support in accordance with conventional techniques. Various automatic synthesizers are commercially available and can be used in accordance with known protocols. (See, for example, Stewart & Young, SOLID PHASE PEPTIDE SYNTHESIS, 2D. ED., Pierce Chemical Co., 1984). Further, individual fragments of rev, tat, and nef can be joined using chemical ligation to produce larger peptides that are still within the bounds of the invention.

In addition, nonclassical amino acids or chemical amino acid analogs can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogs in general. Furthermore, the amino acid can be D (dextrorotary) or L (levorotary).

Alternatively, recombinant DNA technology can be employed wherein a nucleotide sequence which encodes an immunogenic peptide of interest is inserted into an expression vector, transformed or transfected into an appropriate host cell and cultivated under conditions suitable for expression. These procedures are generally known in the art, e.g., as described generally in Sambrook and Russell, supra.

Typically, a nucleic acid sequence encoding the chimeric polypeptide is produced by chemical synthesis or is produced by ligating appropriate fragments to one another. The fragments can be produced chemically, or can be obtained by PCR or isolated from plasmids or other vectors that contain the sequence of interest. The coding sequence can then be provided with appropriate linkers and ligated into expression vectors (e.g., for vaccines and/or for production of reocmbinant protein) commonly available in the art, and the vectors used to transform suitable hosts to produce the desired fusion protein. A number of such vectors and suitable host systems are available. For expression of the fusion proteins, the coding sequence will be provided with operably linked start and stop codons, promoter and terminator regions and usually a replication system to provide an expression vector for expression in the desired cellular host. For example, for production of recombinant protein, promoter sequences compatible with bacterial hosts are provided in plasmids containing convenient restriction sites for insertion of the desired coding sequence. The resulting expression vectors are transformed into suitable bacterial hosts. Of course, yeast, insect or mammalian cell hosts may also be used, employing suitable vectors and control sequences (see, e.g., Sambrook and Russell, supra).

Many conservative variations of the nucleic acid sequences can be used to generate an essentially identical polypeptide. For example, due to the degeneracy of the genetic code, “silent substitutions” (i.e., substitutions of a nucleic acid sequence which do not result in an alteration in an encoded polypeptide) are an implied feature of every nucleic acid sequence which encodes an amino acid. Often, the sequences used in constructing a chimeric gene of the invention employ substitutions based on codon usage frequencies. For example, the codon usage typical of lentiviruses, which is biased for adenine and thymidine at the third codon position, reduces the translation efficiency in mammalian cells (Andre et al., J Virol, 72:1497-1503 (1998); Haas et al., Curr Biol, 6:315-324 (1996)). Thus, appropriate codons to optimize mammalian expression can be substituted for the lentivirus codons.

Modifications to rev, tat, and nef nucleic acid and polypeptide sequences incorporated into the chimeric molecules, particularly those which result in a change the amino acid sequence, are evaluated by routine screening techniques to ensure that the chimeric proteins retain immunogenicity. For instance, the ability of the peptide to induce a CD8+ response can be determined using methodology such as cytotoxic T cell assays or direct quantification of antigen-specific T cells by staining with Fluorescein-labeled HLA tetrameric complexes (Altman, J. D. et al., Proc. Natl. Acad. Sci. USA 90:10330, 1993; Altman, J. D. et al., Science 274:94, 1996). Other assays include staining for intracellular lymphokines, and γ-interferon release assays or ELISPOT assays. Similarly, the ability of the peptide to stimulate CD4+ cells can also be determined, e.g., by T-cell proliferation assays.

Typically, suitable modified rev, tat, and nef polypeptides, or fragments of the polypeptides, that can be included as components of the chimeric molecules have about 80% amino acid sequence identity, optionally about 75%, 80%, 85%, 90%, or 95-98% amino acid sequence identity to a known rev, tat, or nef protein sequence over a comparison window of about 20 amino acids, optionally about 25, 30, or, 50-100 amino acids, or the length of the entire protein. The sequence can be compared and aligned for maximum correspondence over a comparison window, or designated region as measured using one of the following sequence comparison algorithms or by manual alignment and visual inspection. For purposes of this patent, percent amino acid identity is determined by the default parameters of BLAST.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The comparison window includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

One example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Cross-Reactive Binding to Antibodies

Rev, tat, nef chimeric polypeptides can also be identified by the abilitiy to cross-react with antibodies, preferably polyclonal antibodies, that bind to known rev, tat, and nef, polypeptides. A rev, tat, nef chimeric polypeptide can be tested for cross-reactivity as a chimeric molecule or can be tested as using specific fragments of the chimeric molecule that correspond to the rev, tat, or nef domains of the fusion proteins.

Polyclonal antibodies are generated using methods well known to those of ordinary skill in the art (see, e.g., Coligan, Current Protocols in Immunology (1991); Harlow & Lane, Antibodies, A Laboratory Manual (1988)). Those rev, tat, nef proteins that are immunologically cross-reactive binding proteins can then be detected by a variety of assay methods. For descriptions of various formats and conditions that can be used, see, e.g., Methods in Cell Biology: Antibodies in Cell Biology, volume 37 (Asai, ed. 1993), Coligan, supra, and Harlow & Lane, supra.

Useful immunoassay formats include assays where a sample protein is immobilized to a solid support. For example, a cross-reactive rev, tat, nef fusion protein can be identified using an immunoblot analysis such as a western blot. The western blot technique generally comprises electrophoresing a sample comprising a rev-tat-nef chimeric polypeptide on a gel, transferring the separated proteins to a suitable solid support, (such as a nitrocellulose filter, a nylon filter, or derivatized nylon filter), and incubating the sample with antibodies that bind to known rev, tat, or nef polypeptides. The antibodies then specifically bind to cross-reactive rev, tat, or nef polypeptides on the solid support. The antibodies may be directly labeled or alternatively may be subsequently detected using labeled antibodies (e.g., labeled sheep anti-mouse antibodies) that specifically bind to the rev, tat, or nef antibodies. Other immunoblot assays, such as dot blots, are also useful for identifying rev, tat, and nef chimeric molecules suitable for use in the invention.

Using this methodology under designated immunoassay conditions, immunologically cross-reactive rev, tat, and nef sequences in the chimeric fusion proteins that bind to a particular rev, tat, or nef antibody at least two times the background or more, typically more than 10 times background, and do not substantially bind in a significant amount to other proteins present in the sample can be identified.

Immunoassays in the competitive binding format can also be used for crossreactivity determinations. For example, polyclonal antisera that have been generated to a known, rev, tat, or nef polypeptide, e.g., HIV-1 rev, tat, or nef can be used. The antisera can be immobilized to a solid support. The ability of added rev, tat, or nef chimeric proteins (or fragments of the rev, tat, or nef chimeric proteins) to compete for binding with known rev, tat, or nef polypeptides is analyzed by comparing the binding to a standard curve generated using the known polypeptide. The crossreactivity for the proteins is calculated, using standard calculations.

In order to make this comparison, the standard protein and test protein are each assayed at a wide range of concentrations and the amount of each protein required to inhibit 50% of the binding is determined. If the amount of the test rev, tat, and nef chimeric protein, or fragment of the chimeric protein, required to inhibit 50% of binding is less than 10 times the amount of the standard protein that is required to inhibit 50% of binding, then test rev, tat, nef protein is said to specifically bind to the polyclonal antibodies generated to the known rev, tat, or nef immunogen.

Rev, tat, and nef Sequences

The rev sequences included in the chimeric polynucleotides and polypeptides can be modified by deleting specific regions, for example, the nuclear localization sequence (NLS) and/or RNA binding domain of Rev. Deletion of these regions thereby prevents nuclear localization of a chimeric polypeptide containing rev and, when the RNA binding domain is deleted, prevents binding to its recognition sequence. Functional disruption can also be achieved by deleting and/or altering specific amino acids within the functional domains.

Tat sequences can be similarly altered to prevent function. For example, tat can be engineered to delete its nuclear localization sequence and/or RNA binding domain. In many embodiments, the RNA binding domains and NLS regions of both rev and tat are deleted or disrupted to prevent such activities in a chimeric peptide of the invention.

Nef includes a myristylation site that is required for translocation of Nef to the cellular membrane and downregulation of the CD4+ and MHC-I molecules. This sequence is an important determination of viral pathogenicity. The myristylation sequence can be deleted or mutagenized to prevent translocation of a chimeric protein comprising nef to the cellular membrane.

The ret, nef, and tat nucleic acid sequences can be included in the construct in any order. The protein-encoding regions can also be dispersed. For example, one or more of the proteins can be divided into an N-terminal part and a C-terminal part. These two parts can then be separated by intervening regions of the other proteins. The fragments or regions of the rev, tat, and nef polypeptide sequences included in the chimeric constructs can vary in size from the full-length polypeptide to fragments of the full-length polypeptide sequences. For example, rev, tat, or nef fragments of about 20, 25, 50, 75, 100, 125, 150, 175, or 200, or 250 amino acids in length can be incorporated into the chimeric polypeptides.

Attenuated Recombinant Viral Vaccines

Attenuated recombinant poxviruses that express retrovirus-specific epitopes are typically used in this invention. Attenuated viruses are modified from their wildtype virulent form to be either symptomless or weakened when infecting humans. Typically, the genome of the virus is defective in respect of a gene essential for the efficient production or essential for the production of infectious virus. The mutant virus acts as a vector for an immunogenic retroviral protein by virtue of the virus encoding foreign DNA. This provokes or stimulates a cell-mediated CD8⁺ response.

The virus is then introduced into a human vaccinee by standard methods for vaccination of live vaccines. A live vaccine of the invention can be administered at, for example, about 10⁴-10⁸ organisms/dose, or 10⁶ to 10⁹ pfu per dose. Actual dosages of such a vaccine can be readily determined by one of ordinary skill in the field of vaccine technology.

The selection of the virus is not critical. Examples of viral expression vectors include adenoviruses as described in M. Eloit et al, “Construction of a Defective Adenovirus Vector Expressing the Pseudorabies Virus Glycoprotein gp50 and its Use as a Live Vaccine”, J. Gen. Virol., 71(10):2425-2431 (October, 1990).), adeno-associated viruses (see, e.g., Samulski et al., J. Virol. 61:3096-3101 (1987); Samulski et al., J. Virol. 63:3822-3828 (1989)), papillomavirus, Epstein Barr virus (EBV) and Rhinoviruses (see, e.g., U.S. Pat. No. 5,714,374). Human parainfluenza viruses are also reported to be useful, especially JS CP45 HPIV-3 strain. The viral vector may be derived from herpes simplex virus (HSV) in which, for example, the gene encoding glycoprotein H (gH) has been inactivated or deleted. Other suitable viral vectors include retroviruses (see, e.g., Miller, Human Gene Ther. 1:5-14 (1990); Ausubel et al., Current Protocols in Molecular Biology). Other viral expression vectors that can be used as vaccines include alphaviruses, such as Venezuelan equine encephalomyelitis virus (see, e.g, U.S. Pat. Nos. 5,643,576 and 6,296,854).

The poxviruses are often used in this invention. There are a variety of attenuated poxviruses that are available for use as a vaccine against HIV. These include attenuated vaccinia virus, cowpox virus and canarypox virus. In brief, the basic technique of inserting foreign genes into live infectious poxvirus involves a recombination between pox DNA sequences flanking a foreign genetic element in a donor plasmid and a homologous sequences present in the rescuing poxvirus as described in Piccini et al., Methods in Enzymology 153, 545-563 (1987). More specifically, the recombinant poxviruses are constructed in two steps known in the art and analogous to the methods for creating synthetic recombinants of poxviruses such as the vaccinia virus and avipox virus described in U.S. Pat. Nos. 4,769,330, 4,722,848, 4,603,112, 5,110,587, and 5,174,993, the disclosures of which are incorporated herein by reference.

First, the DNA gene sequence encoding an antigenic sequence such as a known T-cell epitope is selected to be inserted into the virus and is placed into an E. coli plasmid construct into which DNA homologous to a section of DNA of the poxvirus has been inserted. Separately, the DNA gene sequence to be inserted is ligated to a promoter. The promoter-gene linkage is positioned in the plasmid construct so that the promoter-gene linkage is flanked on both ends by DNA homologous to a DNA sequence flanking a region of pox DNA containing a nonessential locus. The resulting plasmid construct is then amplified by growth within E. coli bacteria.

Second, the isolated plasmid containing the DNA gene sequence to be inserted is transfected into a cell culture, e.g. chick embryo fibroblasts, along with the poxvirus. Recombination between homologous pox DNA in the plasmid and the viral genome respectively gives a poxvirus modified by the presence, in a nonessential region of its genome, of foreign DNA sequences.

Attenuated recombinant pox viruses are a preferred vaccine. A detailed review of this technology is found in U.S. Pat. No. 5,863,542, which is incorporated by reference herein. These viruses are modified recombinant viruses having inactivated virus-encoded genetic functions so that the recombinant virus has attenuated virulence and enhanced safety. The functions can be non-essential, or associated with virulence. The poxvirus is generally a vaccinia virus or an avipox virus, such as fowlpox virus and canarypox virus. The viruses are generated using the general strategy outlined above and in U.S. Pat. No. 5,863,542.

Representative examples of recombinant pox viruses include ALVAC, TROVAC, NYVAC, and vCP205 (ALVAC-MN120TMG). These viruses were deposited under the terms of the Budapest Treaty with the American Type Culture Collection (ATCC), 12301 Parklawn Drive, Rockville, Md., 20852, USA: NYVAC under ATCC accession number VR-2559 on Mar. 6, 1997; vCP205 (ALVAC-MN120TMG) under ATCC accession number VR-2557 on Mar. 6, 1997; TROVAC under ATCC accession number VR-2553 on Feb. 6, 1997 and, ALVAC under ATCC accession number VR-2547 on Nov. 14, 1996.

NYVAC is a genetically engineered vaccinia virus strain generated by the specific deletion of eighteen open reading frames encoding gene products associated with virulence and host range. NYVAC is highly attenuated by a number of criteria including: i) decreased virulence after intracerebral inoculation in newborn mice, ii) inocuity in genetically (nu⁺/nu⁺) or chemically (cyclophosphamide) immunocompromised mice, iii) failure to cause disseminated infection in immunocompromised mice, iv) lack of significant induration and ulceration on rabbit skin, v) rapid clearance from the site of inoculation, and vi) greatly reduced replication competency on a number of tissue culture cell lines including those of human origin.

TROVAC refers to an attenuated fowlpox that was a plaque-cloned isolate derived from the FP-1 vaccine strain of fowlpox virus which is licensed for vaccination of 1 day old chicks.

ALVAC is an attenuated canarypox virus-based vector that was a plaque-cloned derivative of the licensed canarypox vaccine, Kanapox (Tartaglia et al., 1992). ALVAC has some general properties which are the same as some general properties of Kanapox. ALVAC-based recombinant viruses expressing extrinsic immunogens have also been demonstrated efficacious as vaccine vectors. This avipox vector is restricted to avian species for productive replication. On human cell cultures, canarypox virus replication is aborted early in the viral replication cycle prior to viral DNA synthesis. Nevertheless, when engineered to express extrinsic immunogens, authentic expression and processing is observed in vitro in mammalian cells and inoculation into numerous mammalian species induces antibody and cellular immune responses to the extrinsic immunogen and provides protection against challenge with the cognate pathogen.

NYVAC, ALVAC and TROVAC have also been recognized as unique among all poxviruses in that the National Institutes of Health (“NIH”)(U.S. Public Health Service), Recombinant DNA Advisory Committee, which issues guidelines for the physical containment of genetic material such as viruses and vectors, i.e., guidelines for safety procedures for the use of such viruses and vectors which are based upon the pathogenicity of the particular virus or vector, granted a reduction in physical containment level: from BSL2 to BSL1. No other poxvirus has a BSL1 physical containment level. Even the Copenhagen strain of vaccinia virus-the common smallpox vaccine-has a higher physical containment level; namely, BSL2. Accordingly, the art has recognized that NYVAC, ALVAC and TROVAC have a lower pathogenicity than any other poxvirus.

Another attenuated poxvirus of preferred use for this invention is Modified Vaccinia virus Ankara (MVA), which acquired defects in its replication ability in humans as well as most mammalian cells following over 500 serial passages in chicken fibroblasts (see, e.g., Mayr et al., Infection 3:6-14 (1975); Carrol, M. and Moss, B. Virology 238:198-211 (1997)). MVA retains its original immunogenicity and its variola-protective effect and no longer has any virulence and contagiousness for animals and humans. As in the case of NYVAC or ALVAC, expression of recombinant protein occurs during an abortive infection of human cells, thus providing a safe, yet effective, delivery system for foreign antigens.

Nucleic Acid Vaccines

Nucleic acid vaccines, preferably DNA vaccines may also be used in the invention. Preferably, the nucleic acid vaccines is administered in a regimen that also comprises administration of a viral vaccine. Nucleic acid vaccines as defined herein, typically plasmid expression vectors that are not encapsidated in a viral particle. The nucleic acid vaccine is directly introduced into the cells of the individual receiving the vaccine regimen. This approach is described, for instance, in Wolff et. al., Science 247:1465 (1990) as well as U.S. Pat. Nos. 5,580,859; 5,589,466; 5,804,566; 5,739,118; 5,736,524; 5,679,647; and WO 98/04720. Examples of DNA-based delivery technologies include, “naked DNA”, facilitated (bupivicaine, polymers, peptide-mediated) delivery, and cationic lipid complexes or liposomes. The nucleic acids can be administered using ballistic delivery as described, for instance, in U.S. Pat. No. 5,204,253 or pressure (see, e.g., U.S. Pat. No. 5,922,687). Using this technique, particles comprised solely of DNA are administered, or in an alternative embodiment, the DNA can be adhered to particles, such as gold particles, for administration.

As is well known in the art, a large number of factors can influence the efficiency of expression of antigen genes and/or the immunogenicity of DNA vaccines. Examples of such factors include the reproducibility of inoculation, construction of the plasmid vector, choice of the promoter used to drive antigen gene expression and stability of the inserted gene in the plasmid.

Any of the conventional vectors used for expression in eukaryotic cells may be used for directly introducing DNA into tissue. Expression vectors containing regulatory elements from eukaryotic viruses are typically used in eukaryotic expression vectors, e.g., SV40 CMB vectors. Other exemplary eukaryotic vectors include pMSG, pAV009/A+, pMTO10/A+, pMAMneo-5, and any other vector allowing expression of proteins under the direction of such promoters as the SV40 early promoter, SV40 later promoter, metallothionein promoter, human cytomegalovirus promoter, murine mammary tumor virus promoter, Rous sarcoma virus promoter, polyhedrin promoter, or other promoters shown effective for expression in eukaryotic cells.

Therapeutic quantities of plasmid DNA can be produced for example, by fermentation in E. coli, followed by purification. Aliquots from the working cell bank are used to inoculate growth medium, and grown to saturation in shaker flasks or a bioreactor according to well known techniques. Plasmid DNA can be purified using standard bioseparation technologies such as solid phase anion-exchange resins. If required, supercoiled DNA can be isolated from the open circular and linear forms using gel electrophoresis or other methods.

Purified plasmid DNA can be prepared for injection using a variety of formulations. The simplest of these is reconstitution of lyophilized DNA in sterile phosphate-buffer saline (PBS). This approach, known as “naked DNA,” is particularly suitable for intramuscular (IM) or intradermal (ID) administration.

To maximize the immunotherapeutic effects of minigene DNA vaccines, alternative methods for formulating purified plasmid DNA may be desirable. A variety of methods have been described, and new techniques may become available. Cationic lipids can also be used in the formulation (see, e.g., as described by WO 93/24640; Mannino & Gould-Fogerite, BioTechniques 6(7): 682 (1988); U.S. Pat. No. 5,279,833; WO 91/06309; and Felgner, et al., Proc. Nat'l Acad. Sci. USA 84:7413 (1987). In addition, glycolipids, fusogenic liposomes, peptides and compounds referred to collectively as protective, interactive, non-condensing compounds (PINC) could also be complexed to purified plasmid DNA to influence variables such as stability, intramuscular dispersion, or trafficking to specific organs or cell types.

Polypeptide Vaccines

The chimeric rev, tat, and nef polypeptides of the invention can also be administered as vaccines. Theses polypeptides can be produced by chemical synthesis or by recombinant DNA technology as described above.

The chimeric rev, tat, and nef contstructs can also be administered as peptide. The peptide can be administered with a variety of agents, e.g., a carrier.

There are a number of strategies for amplifying an immunogen's effectiveness, particularly as related to the art of vaccines. These include strategies whereby an immunogenic peptide may be directly modified to enhance immunogenicity or physical properties such as stability. For example, cyclization or circularization of a peptide can increase the peptide's antigenic and immunogenic potency. See, e.g., U.S. Pat. No. 5,001,049 which is incorporated by reference herein.

The immunogenicity of the chimeric rev, tat, and nef proteins may also be modulated by coupling to fatty acid moieties to produce lipidated peptides. Convenient fatty acid moieties include glycolipid analogs, N-palmityl-S-(2RS)-2,3-bis-(palmitoyloxy)propyl-cysteinyl-serine (PAM3 Cys-Ser), N-palmityl-S-[2,3 bis (paInitoyloxy)-(2RS)-propyl-[R]-cysteine (TPC), tripalmitoyl-S-glycerylcysteinlyseryl-serine (P₃CSS), or adipalmityl-lysine moiety

The polypeptides may also be conjugated to a lipidated amino acid, such as an octadecyl ester of an aromatic acid, such as tyrosine, including actadecyl-tryrosine (OTH).

Carriers may also be used with the polypeptide vaccines. Carriers aree well known in the art, and include, e.g., thyroglobulin, albumins such as human serum albumin, tetanus toxoid, polyamino acids such as poly L-lysine, poly L-glutamic acid, i and the like. The vaccines can contain a physiologically tolerable (i.e., acceptable) diluent such as water, or saline, preferably phosphate buffered saline. The vaccines also typically include an adjuvant. Adjuvants such as incomplete Freund's adjuvant, aluminum phosphate, aluminum hydroxide, or alum are examples of materials well known in the art.

Characterization of the Immune Response in Vaccinated Individuals

The vaccine regimen can be delivered to individuals at risk for infection with HIV or to patients who are infected with the virus. In order to assess the efficacy of the vaccine, the immune response can be assessed by measuring the induction of CD4⁺, CD8⁺, and antibody responses to particular epitopes. Moreover, viral titer can be measured in patients treated with the vaccine who are already infected. These parameters can be measured using techniques well known to those of skill in the art. Examples of such techniques are described below.

CD4⁺ T Cell Counts

To assess the effectiveness of the vaccine combination in a recipient and to monitor the immune system of a patient already infected with the virus who is a candidate for treatment with the vaccine regimen, it is important to measure CD4⁺ T cell counts. A detailed description of this procedure was published by Janet K. A. Nicholson, Ph.D et al. 1997 Revised Guidelines for Performing CD4+ T-Cell Determinations in Persons Infected with Human Immunodeficiency Virus (HIV) in The Morbidity and Mortality Weekly Report, 46(RR-2):[inclusive page numbers], Feb. 14, 1997. Centers for Disease Control.

In brief, most laboratories measure absolute CD4⁺ T-cell levels in whole blood by a multi-platform, three-stage process. The CD4⁺ T-cell number is the product of three laboratory techniques: the white blood cell (WBC) count; the percentage of WBCs that are lymphocytes (differential); and the percentage of lymphocytes that are CD4⁺ T-cells. The last stage in the process of measuring the percentage of CD4⁺ T-lymphocytes in the whole-blood sample is referred to as “immunophenotyping by flow cytometry.

Immunophenotyping refers to the detection of antigenic determinants (which are unique to particular cell types) on the surface of WBCs using antigen-specific monoclonal antibodies that have been labeled with a fluorescent dye or fluorochrome (e.g., phycoerythrin [PE] or fluorescein isothiocyanate [FITC]). The fluorochrome-labeled cells are analyzed by using a flow cytometer, which categorizes individual cells according to size, granularity, fluorochrome, and intensity of fluorescence. Size and granularity, detected by light scattering, characterize the types of WBCs (i.e., granulocytes, monocytes, and lymphocytes). Fluorochrome-labeled antibodies distinguish populations and subpopulations of WBCs.

Systems for measuring CD4⁺ cells are commercially available. For example Becton Dickenson's FACSCount System automatically measure absolutes CD4⁺, CD8⁺, and CD3⁺0 T lymphocytes. It is a self-contained system, incorporating instrument, reagents, and controls.

A successful increase of CD4⁺ cell counts would be a 2× or higher increase in the number of CD4⁺ cells.

Measurements of CD8⁺ Responses

CD8⁺ T-cell responses may be measured, for example, by using tetramer staining of fresh or cultured PBMC, ELISPOT assays or by using functional cytotoxicity assays, which are well-known to those of skill in the art. For example, a functional cytotoxicity assay can be performed as follows. Briefly, peripheral blood lymphocytes from patients are cultured with HIV peptide epitope at a density of about five million cells/ml. Following three days of culture, the medium is supplemented with human IL-2 at 20 units/ml and the cultures are maintained for four additional days. PBLs are centrifuged over Ficoll-Hypaque and assessed as effector cells in a standard ⁵¹Cr-release assay using U-bottomed microtiter plates containing about 10⁴ target cells with varying effector cell concentrations. All cells are assayed twice. Autologous B lymphoblastoid cell lines are used as target cells and are loaded with peptide by incubation overnight during ⁵¹Cr labeling. Specific release is calculated in the following manner: (experimental release-spontaneous release)/(maximum release-spontaneous release)×100. Spontaneous release is generally less than 20% of maximal release with detergent (2% Triton X-100) in all assays. A successful CD8⁺ response occurs when the induced cytolytic activity is above 10% of controls.

Another measure of CD8⁺ responses provides direct quantification of antigen-specific T cells by staining with Fluorescein-labeled HLA tetrameric complexes (Altman, J. D. et al., Proc. Natl. Acad. Sci. USA 90:10330, 1993; Altman, J. D. et al., Science 274:94, 1996). Other assays include staining for intracellular lymphokines, and γ-interferon release assays or ELISPOT assays. Tetramer staining, intracellular lymphokine staining and ELISPOT assays all are sensitive measures of T cell response (Laivani, A. et al., J. Exp. Med. 186:859, 1997; Dunbar, P. R. et al., Curr. Biol. 8:413, 1998; Murali-Krishna, K. et al., Immunity 8:177, 1998).

Viral Titer

There are a variety of ways to measure viral titer in a patient. A review of the state of this art can be found in the Report of the NIH To Define Principles of Therapy of HIV Infection as published in the; Morbidity and Mortality Weekly Reports, Apr. 24, 1998, Vol 47, No. RR-5, Revised Jun. 17, 1998. It is known that HIV replication rates in infected persons can be accurately gauged by measurement of plasma HIV concentrations.

HIV RNA in plasma is contained within circulating virus particles or virions, with each virion containing two copies of HIV genomic RNA. Plasma HIV RNA concentrations can be quantified by either target amplification methods (e.g., quantitative RT polymerase chain reaction [RT-PCR], Amplicor HIV Monitor assay, Roche Molecular Systems; or nucleic acid sequence-based amplification, [NASBA®], NucliSens™ HIV-1 QT assay, Organon Teknika) or signal amplification methods (e.g., branched DNA [bDNA], Quantiplex™ HIV RNA bDNA assay, Chiron Diagnostics). The bDNA signal amplification method amplifies the signal obtained from a captured HIV RNA target by using sequential oligonucleotide hybridization steps, whereas the RT-PCR and NASBA® assays use enzymatic methods to amplify the target HIV RNA into measurable amounts of nucleic acid product. Target HIV RNA sequences are quantitated by comparison with internal or external reference standards, depending upon the assay used.

Measurement of Antibody Response

The ability of a patient to mount a B-cell response to the chimeric protein can also be measured. Typically, a serum sample from the patient is assayed for the presence of antibodies after administration of the chimeric rev, tat, and nef polypeptide. Antibodies can be detected using a variety of immunoassays, including competitive and non-competitive formats. Such assays are described in a number of publications, e.g., CURRENT PROTOCOLS IN IMMUNOLOGY, Coligan et al., Eds., 1995-2001, John Wiley & Sons, Inc.; Harlow & Lane, ANTIBODIES: A LABORATORY MANUAL, Cold Spring Harbor Laboratory Press, 1988; and Harlow & Lane, USING ANTIBODIES: A LABORATORY MANUAL, Cold Spring Harbor Laboratory Press, 1999.

Formulation of Vaccines and Administration

The administration procedure for recombinant virus or DNA is not critical. Vaccine compositions (e.g., compositions containing the poxvirus recombinants or DNA) can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular patient, and the route of administration.

The vaccines can be administered prophylactically or therapeutically. In prophylactic administration, the vaccines are administered in an amount sufficient to induce CD8⁺ and CD4⁺, or antibody, responses. In therapeutic applications, the vaccines are administered to a patient in an amount sufficient to elicit a therapeutic effect, i.e., a CD8⁺, CD4⁺, and/or antibody response to the HIV-1 antigens or epitopes encoded by the vaccines that cures or at least partially arrests or slows symptoms and/or complications of HIV infection. An amount adequate to accomplish this is defined as “therapeutically effective dose.” Amounts effective for this use will depend on, e.g., the particular composition of the vaccine regimen administered, the manner of administration, the stage and severity of the disease, the general state of health of the patient, and the judgment of the prescribing physician.

The vaccine can be administered in any combination, the order is not critical. In some instances, for example, a DNA HIV vaccine is administered to a patient more than once followed by delivery of one or more administrations of the recombinant pox virus vaccine. The recombinant viruses are typically administered in an amount of about 10⁴ to about 10⁹ pfu per inoculation; often about 10⁴ pfu to about 10⁶ pfu. Higher dosages such as about 10⁴ pfu to about 10¹⁰ pfu, e.g., about 10⁵ pfu to about 10⁹ pfu, or about 10⁶ pfu to about 10⁸ pfu, can also be employed. For example, a NYVAC-HIV vaccine can be inoculated by the intramuscular route at a dose of about 10⁸ pfu per inoculation, for a patient of 170 pounds.

Suitable quantities of DNA vaccine, e.g., plasmid or naked DNA can be about 1 μg to about 100 mg, preferably 0.1 to 10 mg, but lower levels such as 0.1 to 2 mg or 1-10 μg can be employed. For example, an HIV DNA vaccine, e.g., naked DNA or polynucleotide in an aqueous carrier, can be injected into tissue, e.g., intramuscularly or intradermally, in amounts of from 10 μl per site to about 1 ml per site. The concentration of polynucleotide in the formulation is from about 0.1 μg/ml to about 20 mg/ml.

The vaccines may be delivered in a physiologically compatible solution such as sterile PBS in a volume of, e.g., one ml. The vaccines can also be lyophilized prior to delivery. As well known to those in the art, the dose may be proportional to weight.

The compositions included in the vaccine regimen of the invention can be co-administered or sequentially administered with other immunological, antigenic or vaccine or therapeutic compositions, including an adjuvant, a chemical or biological agent given in combination with or recombinantly fused to an antigen to enhance immunogenicity of the antigen. Additional therapeutic products can include biological response modifiers such as cytokines or co-stimulatory agents, e.g., interleukin-2 (IL-2) or CD40 ligand in an amount that is sufficient to further potentiate the CD8⁺ and CD4⁺ T-cell responses.

Other compositions that can be administered with the vaccines of the current invention include purified antigens from the immunodeficiency virus or proteins obtained from the expression of such antigens by a second recombinant vector system. For example, additional compositions can include vaccines, such as nucleic-acid based vaccines, that encode other HIV proteins, for instance structural proteins, e.g., gag, pol, and env. Administration of these additional agents can occur before, after, or concurrently with adminsitration of the vaccines comprising chimeric rev, tat, and nef.

Examples of adjuvants which also may be employed include Freund's complete adjuvant and MPL-TDM adjuvant (monophosphoryl Lipid A, synthetic trehalose dicorynomycolate). Again, co-administration is performed by taking into consideration such known factors as the age, sex, weight, and condition of the particular patient, and, the route of administration.

The peptide, viral and DNA vaccines can additionally be complexed with other components such as lipids, peptides, polypeptides and carbohydrates for delivery. Such formulations are known in the art, see, e.g., Remington's Pharmaceutical Sciences, 17^{th} Edition, A. Gennaro, Editor, Mack Publising Co., Easton, Pa., 1985)

The DNA vaccines are administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997). The vectors can also be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

Vaccines may be delivered via a variety of routes. Typical delivery routes include parenteral administration, e.g., intradermal, intramuscular or subcutaneous delivery. Other routes include oral administration, intranasal, and intravaginal routes. For DNA vaccines in particular, the vaccines can be delivered to the interstitial spaces of tissues of an individual (Felgner et al., U.S. Pat. Nos. 5,580,859 and 5,703,055). Administration of DNA vaccines to muscle is also a frequently used method of administration, as is intradermal and subcutaneous injections and transdermal administration. Transdermal administration, such as by iontophoresis, is also an effective method to deliver nucleic acid vaccines to muscle. Epidermal administration of expression vectors of the invention can also be employed. Epidermal administration involves mechanically or chemically irritating the outermost layer of epidermis to stimulate an immune response to the irritant (Carson et al., U.S. Pat. No. 5,679,647).

The vaccines can also be formulated for administration via the nasal passages. Formulations suitable for nasal administration, wherein the carrier is a solid, include a coarse powder having a particle size, for example, in the range of about 10 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active ingredient. For further discussions of nasal administration of AIDS-related vaccines, references are made to the following patents, U.S. Pat. Nos. 5,846,978, 5,663,169, 5,578,597, 5,502,060, 5,476,874, 5,413,999, 5,308,854, 5,192,668, and 5,187,074.

Examples of vaccine compositions of use for the invention include liquid preparations, for orifice, e.g., oral, nasal, anal, vaginal, etc. administration, such as suspensions, syrups or elixirs; and, preparations for parenteral, subcutaneous, intradermal, intramuscular or intravenous administration (e.g., injectable administration) such as sterile suspensions or emulsions. In such compositions the recombinant poxvirus, expression product, immunogen, DNA, or modified gp120 or gp160 may be in admixture with a suitable carrier, diluent, or excipient such as sterile water, physiological saline, glucose or the like.

The vaccines can be incorporated, if desired, into liposomes, microspheres or other polymer matrices (Felgner et al., U.S. Pat. No. 5,703,055; Gregoriadis, Liposome Technology, Vols. I to III (2nd ed. 1993), each of which is incorporated herein by reference). Liposomes, for example, which consist of phospholipids or other lipids, are nontoxic, physiologically acceptable and metabolizable carriers that are relatively simple to make and administer.

Liposome carriers may serve to target a particular tissue or infected cells, as well as increase the half-life of the vaccine. Liposomes include emulsions, foams, micelles, insoluble monolayers, liquid crystals, phospholipid dispersions, lamellar layers and the like. In these preparations the vaccine to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes either filled or decorated with a desired immunogen of the invention can be directed to the site of lymphoid cells, where the liposomes then deliver the immunogen(s). Liposomes for use in the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka, et al., Ann. Rev. Biophys. Bioeng. 9:467 (1980), U.S. Pat. Nos. 4,235,871, 4,501,728, 4,837,028, and 5,019,369.

The dosage for an initial immunization generally occurs in a unit dosage range where the lower value is about 1, 5, 50, 500, or 1,000 μg and the higher value is about 10,000; 20,000; 30,000; or 50,000 μg. Dosage values for a human typically range from about 500 μg to about 50,000/g per 70 kilogram patient. Boosting dosages of between about 1.0 μg to about 50,000 μg of peptide pursuant to a boosting regimen over weeks to months may be administered depending upon the patient's response and condition as determined by measuring the specific activity of CTL and HTL obtained from the patient's blood or by measuring antibody response. The dosages, routes of administration, and dose schedules are adjusted in accordance with methodologies known in the art.

The concentration of peptides of the invention in the pharmaceutical formulations for administration as a vaccine can vary widely, i.e., from less than about 0.1%, usually at or at least about 2% to as much as 20% to 50% or more by weight, and will be selected primarily by fluid volumes, viscosities, etc., in accordance with the particular mode of administration selected.

A human unit dose form of the peptide composition is typically included in a pharmaceutical composition that comprises a human unit dose of an acceptable carrier, preferably an aqueous carrier, and is administered in a volume of fluid that is known by those of skill in the art to be used for administration of such compositions to humans (see, e.g., Remington's Pharmaceutical Sciences, supra).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

### EXAMPLES

### Example 1

### Preparation of an SIV retanef Chimeric Gene

The overall composition of the retanef gene is depicted on FIG. 1. To disrupt the functional domains within the tat and nef genes, both genes were split into two segments, and reassorted in the Retanef construct so that the C-terminal part of Nef precedes its N-terminal part and the two are separated by C-terminal part of Tat (FIG. 1). In order to minimize/prevent nuclear localization and RNA-binding of the Retanef protein, the amino acids comprising the nuclear localization sequence (NLS) and RNA-binding domain of Rev (position 40-7) (Henderson et al., J Mol Biol, 274:693-707 (1997); Truant et al., Mol Cell Biol, 19:1210-1217 (1999)) and Tat (position 81-84) (Truant et al., Mol Cell Biol, 19:1210-1217 (1999)) and Tat (position 81-84) (71) were deleted (FIG. 2). The myristylation signal at the N-terminus of the Nef protein, a necessary requirement for translocation of Nef to cellular membrane and downregulation of the CD4 and MHC-I molecules (Hua et al., Virology, 231:231-238 (1997); Sawai et al., J Biol Chem, 270:15307-15314 (1995)), and key for viral pathogenicity in vivo (Aldrovandi et al., J Virol, 72:7032-7039 (1998)), was deleted. In order to preserve putative CTL epitopes, the N- and C-terminus of Nef protein was designed with an overlap of eight amino acids (HRILDIYL). The DNA sequence was modified by the use of different codons in the two overlapping regions to minimize the risk of recombination. The nef sequence was obtained from the SIVmac239 strain and the premature stop codon was mutagenagenized from TAA to GAA. The known SIVmac239 Nef CTL epitopes recognized by infected and vaccinated rhesus macaques (Allen et al., J. Virol., 75:738-749 (2001); Evans et al., J Virol, 74:7400-7410 (2000); Evans et al., Nat.Med., 5:1270-1276 (1999)) are conserved in the Retanef construct.

Because the codon usage of lentiviruses reduces the efficacy of protein translation in mammalian cells (Andre et al., J Virol, 72:1497-1503 (1998); Haas et al., Curr Biol, 6:315-324 (1996)), the DNA sequence of Retanef was designed using appropriate codons for expression in mammalian genes. The DNA sequences of the Tat and Rev were adapted from known SIVmac isolates. The HA.1 epitope tag was added at the C-terminus of the retanef gene to facilitate the detection of the chimeric protein. The RTN DNA was cloned into the pCMV/Kan plasmid under the control of CMV promoter and into the highly attenuated poxvirus vector NYVAC under the control of H6 promoter.

A description of the plasmid construction is provided below.

The sequences of rev and tat genes were derived from the published sequences of SIVmac251 isolates (Genbank accession numbers M19499, M15897, M16125, M24614, X06391, X06393, X06879, Y00283, Y00294, Y00295). The nef gene sequence was designed according to the sequence of SIVmac239 nef with a TAA to GAA mutation in position E92 in order to repair the premature stop codon. The C-terminal part contains the sequence that repaired SIVmac239 nef shares with other SIVmac isolates (SIVML, Swiss-Prot #P11262; SIVM1, #P05862) and differs from the sequence of SIVMK isolate (SwissProt #P05861).

The Retanef construct also includes a sequence that reconstitutes the H6 promoter in the pATIHIVMNT plasmid. Two restriction endonuclase recognition sites for SpeI and SalI were inserted between C-Tat 2 and N-Nef in order to facilitate cloning of additional genes into retanef. An HA. 1 protein tag was attached to the C-terminus to facilitate the detection of the protein using commercially available antibodies.

The retanef gene construct was synthesized by Midland Molecular Biology Group (Midland, Tex.). An SacII/EcORI fragment containing the Retanef construct was cloned into an expression vector derived from the kanamycin-expressing pVR1332 (Vical Inc.) (31) under the control of a CMV promoter. Plasmid preparations of clinical-grade quality were produced by Qiagen (Hilden, Germany). The NruI/XhoI Retanef fragment was cloned into pATIHIVMNT plasmid (Virogenetics, NY) and inserted by homologous recombination into the NYVAC vector to obtain the NYVAC-SIV-rtn recombinant vaccine vp1658, as previously described (Benson et al., J. Virol., 72:4170-4182 (1998)).

### Example 2

### Expression and Cellular Localization of DNA-SIV-rtn and the Recombinant NYVAC-SIV-rtn in Monkey and Human Cells

Expression of the retanef gene was assessed by transfecting the DNA-SIV-rtn construct into Hela cells.

Transfection was performed as follows. Hela-Tat cells were plated at 3×10⁵ cells/per 6 cm-diameter plates and after 16 hrs transfected by the calcium phosphate method (28). For transfection, 1 μg of pCMV/Retanef or pCMV/Gag were used and the amount of transfected DNA was normalized to 2 μg with pMEI8S expression vector obtained from Atsushi Miyajima (DNAX, Palo Alto, Calif.). Control cells were transfected with 2 μg of pME18S DNA. Twenty four hours later, the cells were lysed and the amount of protein was determined. Total cellular protein (30 μg) was electrophoresed on a 10% SDS gel, transferred to a nitrocellulose membrane, and analyzed by Western blotting using an anti-HA antibody, 3F10-HRP (Roche Biochemicals, Indianapolis).

A 55 kDa protein was detected in the DNA-SIV-rtn-transfected cells but not in the mock-transfected cells. Expression of the Retanef protein in the cell lysate of African green monkey-derived Vero cells infected with NYVAC or the nonrecombinant NYVAC vector was also determined using Western blots. The 55 kDa Retanef protein was detected only in cells infected with NYVAC-SIV-rtn.

To determine the subcellular localization of the Retanef protein, Hela cells were transfected with either DNA-SIV-rtn or control DNA-SIV-rtn Gag plasmids and analyzed using an indirect immunofluorescence assay which was performed as described below.

Hela-Tat cells were seeded onto slides (2×10⁵ cells per slide) and transfected the following morning. One-fifth of the above transfection mix was used to transfect the cells on the slides. Twenty-four hours later, cells were washed twice in PBS, fixed at room temperature for 7 minutes in 2% paraformaldehyde in PBS, and incubated for 1 hour at 37° C. in 0.1% Saponin (Sigma). Slides were incubated with a 1:100 dilution of anti-HA antibody (12CA5, Roche Biochemicals) in 10% FCS/PBS for 1 hour at 37° C., washed four times in 0.02% Tween 20/PBS, and were incubated as described for the primary antibody with a 1:1000 dilution of a Cy2-conjugated anti-mouse antibody (Jackson ImmunOResearch, PA). After washing, cells were counterstained with Evan's Blue, washed, and stained with DAPI.

No specific staining was observed in cells transfected with either the control plasmid or the pCMV Gag, whereas cells infected with the DNA-SIV-rtn cells were detected. The Retanef protein localized mainly in the perinuclear and cytoplasmic compartments of the transfected cells and did not appear to accumulate in the nucleus of the transfected cells.

### Example 3

### DNA-SIV-rtn and NYVAC-SIV-rtn are Immunogenic in Naive Rhesus Macaques

It has been shown that priming with DNA-SIV-gag-env (DNA-SIV-ge) followed by boosting with NYVAC-SIV-gag-pol-env (NYVAC-SIV-gpe) vaccine candidate induces high frequency of virus specific CTLs and strong and durable lymphoproliferative responses in rhesus macaques. In order to assess the immunogenicity of the DNA-SIV-rtn vaccine candidate, naive rhesus macaques were immunized with DNA-SIV-rtn by the intramuscular and intradermal routes three times and boosted with a single dose of NYVAC-SIV-rtn at week 25. Immunization was performed as follows.

All animals were colony bred rhesus macaques (Macaca mulatta) obtained from Covance Research Products (Alice, Tex.). Animals were housed and handled in accordance with the standards of the Association for the Assessment and Accreditation of Laboratory Animal Care International (AAALAC International). All rhesus macaques were seronegative for SIV-1, STLV-1, and herpesvirus B prior to the study. Screening for the presence of Mamu-A*01 allele was performed using PCR as described (Knapp et al., Tissue Antigens, 50:657-661 (1997)). DNA immunization was performed as follows: 4 mg of pCMV/Retanef or pCMV/Gag plasmid were administered in a regimen of 4 doses of 0.75 mg of each plasmid injected intramuscularly into 2 sites on each leg and 5 doses of 0.2 mg of each plasmid injected intradermally at 5 different sites in the abdominal area. For NYVAC-SIV-rtn immunization, animals were inoculated intramuscularly with 10⁸ pfu of NYVAC-SIV-rtn per immunization.

Naive rhesus macaques 687, 688, and 820 were immunized with three doses of DNA-SIV-rtn simultaneously by intramuscular and intradermal routes at week 0, 4, and 12, followed by a boost with a single dose of NYVAC-SIV-rtn vaccine given intramuscularly at week 25.

Epitope specific CD3+ CD8+ T lymphphocytes were detected by flow cytometry. Fresh PBMC were stained with anti-human CD3 Ab (PerCP labeled, clone SP34, Pharmingen, San Diego, Calif.), anti-human CD8 (FITC labeled, Becton-Dickinson, San Jose, Calif.), and Mamu-A*01 tetrameric complexes refolded in the presence of a specific peptide (kindly provided by Dr. J. Altman) and conjugated to PE labeled streptavidin (Molecular Probes, Eugene, Oreg.). Samples were analyzed on a FACSCalibur (Becton-Dickinson) and the data are presented as percentage of tetramer positive cells of all CD3+ CD8+ lymphocytes.

Monkey IFN-γ specific ELISPOT kits manufactured by U-Cytech (Utrecht, The Netherlands) were used in order to detect the number of cells producing IFN-γ upon in vitro stimulation. Ninety-six well flat bottom plates were coated with anti-IFN-γ mAb MD-1 overnight at 4° C. and blocked with 2% BSA in PBS for 1 hour at 37° C. About 10⁵ cells per well were loaded in quadruplicates in RPMI-1640 containing 5% human serum and 10 μg per ml of a specific peptide or 5 μg per ml Concanavalin A as a positive control. The plates were incubated overnight at 37° C., 5% CO₂, and developed according to the manufacturer's guidelines (U-Cytech).

CTL response was also measured using a CTL assay cytotoxicity assay. About 5×10⁶ PBMC were cultured with 10 μg/ml of specific peptide for three days, IL-2 (Roche, Indianopolis, Ind.) was added at 40 IU/ml and the cells were cultured for another four days. Twelve hours before the killing assay a second dose of IL-2 at 40 IU/ml was added. The cells were then incubated for 6 hours in various effector to target cell ratios with Mamu-A*01-positive ⁵¹Cr-labeled transformed B cells pulsed overnight with 10 μg per ml of a specific peptide. The killing of cells pulsed with an unrelated peptide in a control experiment was equal to the killing observed in the absence of any peptide.

The analysis of vaccine-induced immune responses showed that two weeks following the last immunization, a response to the immunodominant Mamu-A*01-restricted CTL Tat epitope TTPESANL (Tat28) (4) was detected in both Mamu-A*01 positive animals (687 and 688) by staining with a Tat28-specific tetramer at two weeks following the boost. Cells from the Mamu-A*01-negative control macaque 820 did not stain with the tetramer, demonstrating the specificity and MHC-I restriction of this tetramer molecule.

The functionality of these Tat-specific responses was assayed using IFN-γ ELISPOT. An increase in the number of cells producing IFN-γ following in vitro stimulation with the Tat28 peptide was observed after one week from the last immunization. Furthermore, the cells from both Mamu-A*01-positive vaccinated animals, but not from two Mamu-A*01-positive naive control animals, were able to lyse Tat28 peptide-pulsed ⁵¹Cr-labeled target B-cells following a 7-day expansion in culture in the presence of the Tat28 peptide.

Thus, the use of three different assays demonstrated the immunogenicity of the Retanef-based vaccine in naive rhesus macaques.

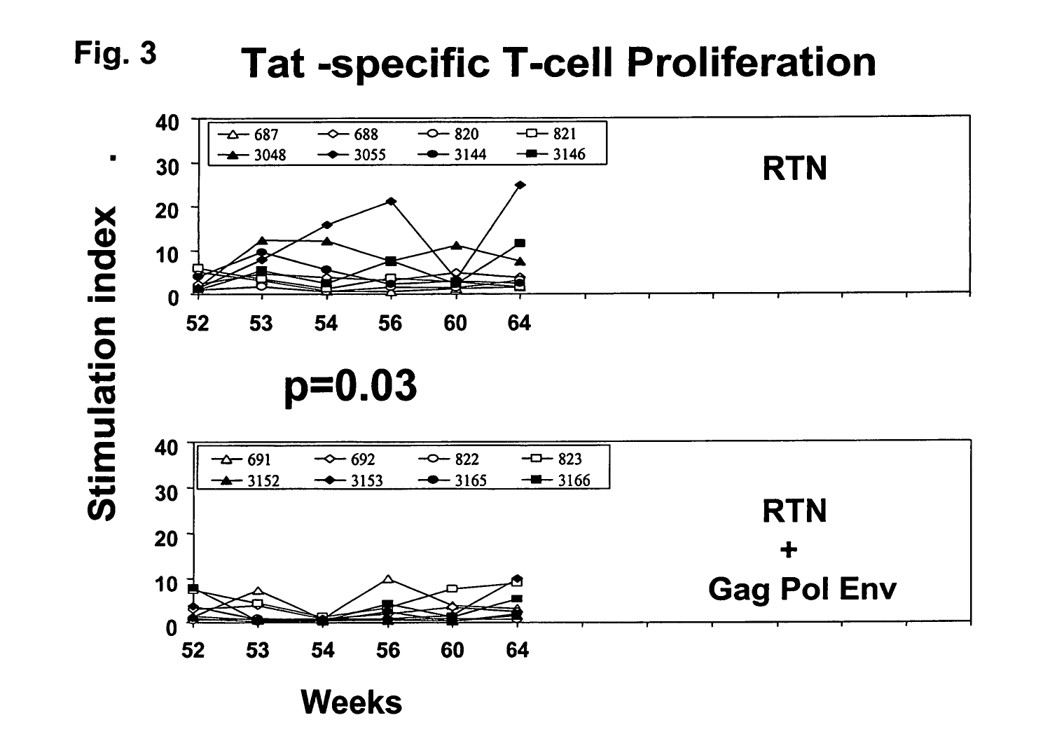

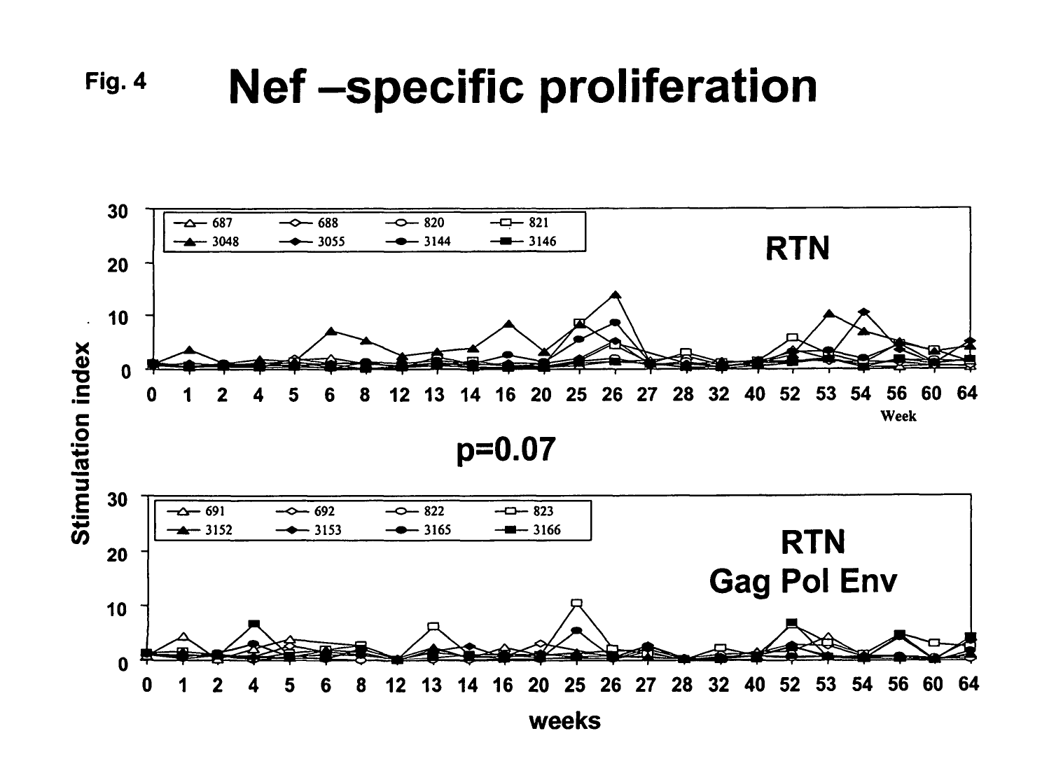

CD4⁺ T-cell proliferative responses to Tat and Nef were also assessed at later time points. Both Tat-specific and Nef-specific T-cell proliferation was observed over 52 weeks after initiation of the vaccine regimen (FIGS. 3 and 4, top panels).

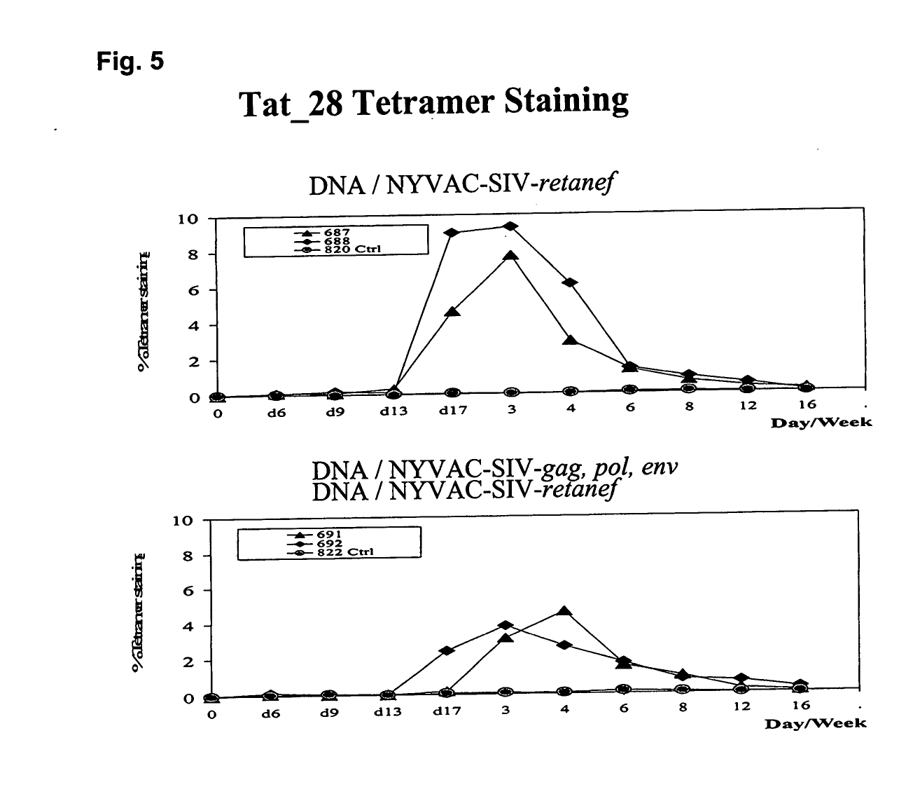

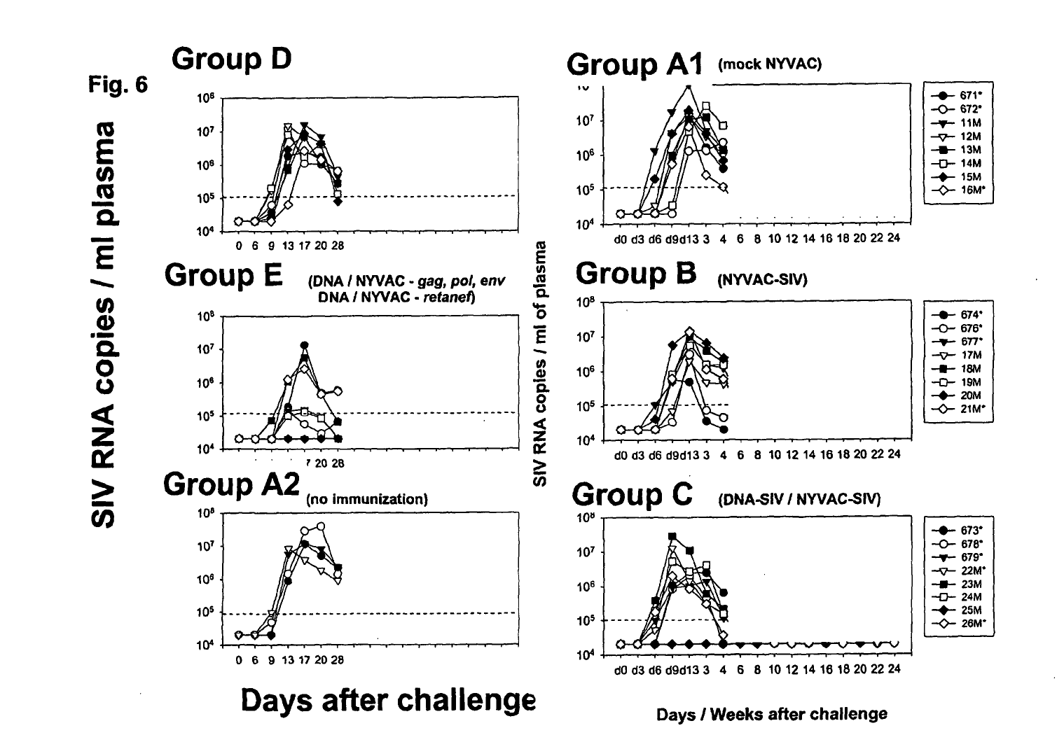

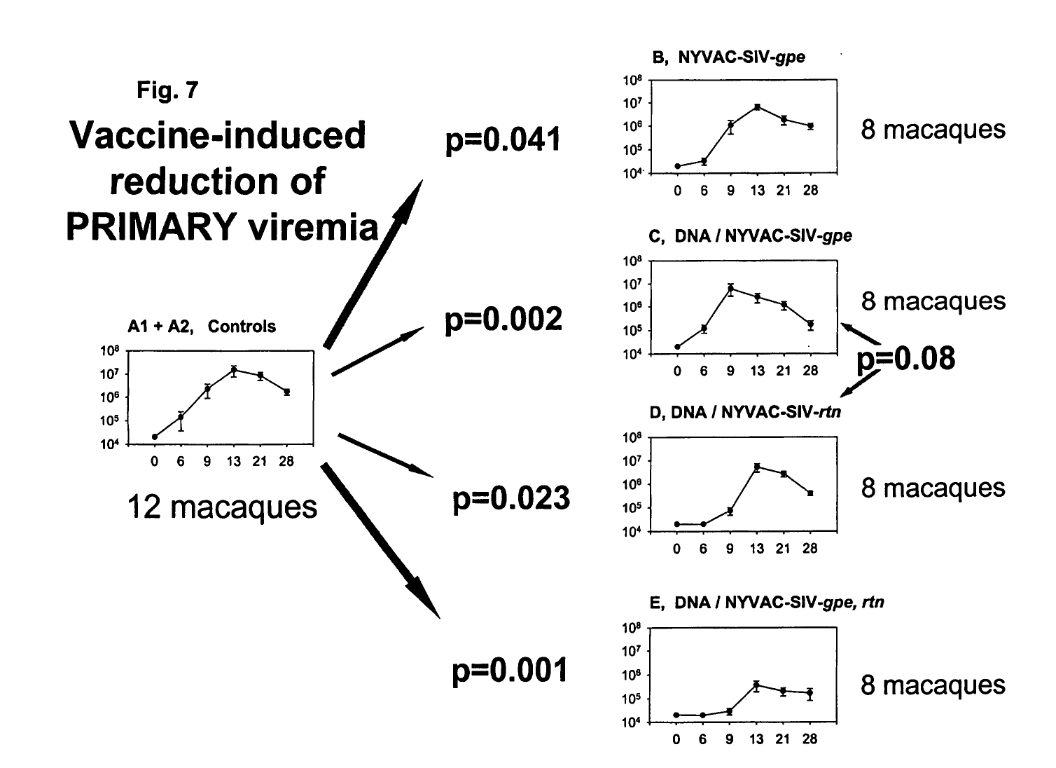

Induction of immune responses to individual antigens in animals vaccinated with DNA/NYVAC-SIV-retanef alone was compared to those vaccinated with both DNA/NYVAC-SIV-retanef and DNA/NYVAC-SIV-gag-pol-env. Animals were vaccinated with DNA/NYVAC-SIV-gag-pol-env (Hel et al., J Immunol 67:7180-91, 2001). The retanef and gag-pol-env DNA constructs were administered at the same time, but at different sites using the DNA/NYVAC regimen previously described (Hel et al., J Immunol 67:7180-91, 2001). The time frame and dose of of administration, i.e., t Tat-specific and Nef-specific CD4+ proliferation was generally reduced in comparison to those animals that were inoculated only with DNA/NYVAC-SIV-retanef (FIGS. 3 and 4, lower panels). Furthermore, tetramer staining with Tat 28 showed that in macaques that were immunized only with the Retanef construct, by day 17, the Tat response was more than 4%, whereas it reached the same level in both animals vaccinated with all antigens by week 3 (FIG. 5). Despite a decrease of the relative amounts of the virus-specific immune response, a significantly lower degree of viremia during acute infection was observed in the primary viremia of macaques immunized with all antigens (group E, FIGS. 6 and 7). These data suggest that vaccination with all of the antigens improved the virological outcome during primary infection and moreover, suggest that sequential administration of retanf with other vaccines, e.g., those inducing responses to the structural proteins, gag, pol, and env can be preferable.

### Example 4

### Adminstratin of DNA-SIV-rtn and NYVAC-SIV-rtn to SIV-Infected Animals

Active immunization of HIV-1-infected HAART-treated individuals may contribute to transient viremia containment in the absence of HAART (Gotch et al., Immunol Rev, 170:173-182 (1999); Hel et al., Nat.Med., 6:1140-1146 (2000)). The immunogenicity of both DNA-SIV-rtn and NYVAC-SIV-rtn vaccine candidates in chronically SIVmac251 infected macaques treated with antiretroviral therapy (ART) was therefore assessed.

Macaques 454, 455, 460, and 541 were infected for 16 months following an intrarectal challenge exposure to a pathogenic SIVmac251 and subjected to antiretroviral therapy for 14 weeks prior to an inoculation with a single dose of either DNA-SIV-rtn (macaques 454 and 455) or DNA-ge (macaques 460 and 541).

Macaques 3075, 3057, and 3077 were exposued intravenously to SIVmac251 virus and became infected and were started on ART 6 months after infection. The macaques were treated with ART for 8 months prior to a single innoculation of NYVAC-SIV-rtn vaccine candidate (macaques 3075 and 3057) or control mock NYVAC (macaque 3077). Antiretroviral therapy consisted of subcutaneous inoculation of 20 mg/kg/day of PMPA [(R)-9-(2-phosphonylmethoypropyl)adenine], oral administration of 2.4 mg/kg/day of Stavudine (d4T) divided into 2 doses daily, and intravenous inoculation of 10 mg/kg/day of Didanosine (DDI) as described previously (Hel et al., Nat.Med., 6:1140-1146 (2000)).

Both macaques inoculated with a single dose of DNA-Retanef given intramuscularly and intradermally (macaques 454 and 455), but none of the macaques immunized with DNA-gag-env (DNA-ge) (macaques 460 and 541) exhibited a three- to sevenfold increase in the frequency of Tat28 tetramer-staining cells in blood over a two weeks period following the vaccination. Likewise, both DNA-ge immunized macaques, but not the DNA-SIV-rtn immunized animals had an increased number of cells staining with a tetramer specific for a Gag epitope Gag181 after immunization (data not shown), demonstrating once again the specificity of staining with the Tat28 tetramer. A three- to fivefold increase in the frequency of Tat28-specific cells was measured in the blood of both the NYVAC-SIV-rtn immunized macaques. Thus, both the DNA-SIV-rtn and NYVAC-SIV-rtn vaccine candidates increased the SIV Tat specific CD8+ T-cell response by several fold in SIVmac251 chronically infected ART-treated animals.

The above examples describe the design, immunogenicity, and safety of novel vaccine candidates expressing retroviral early regulatory genes in both naive and chronically infected macaques treated with antiretroviral therapy. These vaccine candidates were designed with the aims to minimize possible negative affects of Tat and Nef and to ameliorate expression of these antigens in primate cells. Chimeric Retanef was efficiently expressed in both human and monkey cells and did not accumulate in the nucleus of the transfected cells. Furthermore, both the DNA-SIV-rtn and NYVAC-SIV-rtn vaccine candidates were able to expand a virus-specific CD8+ T-cell response to Tat, as measured by direct tetramer staining in the blood of macaques naive or chronically infected with SIVmac251 and treated with antiretroviral therapy. In naive macaques, the increase of frequency in the Tat response was also confirmed using functional assays such as IFN-γ production and cytolytic activity.

The relative immunogenicity of Tat, Rev, and Nef expressed using a DNA plasmid or within a recombinant NYVAC vector can be compared to the known immunogenicity of the individual antigens. For example, the immunogenicity of biologically active Tat versus a Tat toxoid did not appear to differ significantly, and in neither case was notable protection from infection or high viremia was observed following challenge exposure (Pauza et al., Proc. Natl. Acad. Sci. USA, 97:3515-3519 (2000); Rappaport et al., J Leukoc Biol, 65:458-465 (1999)) (J. Shiver, personal communication). This suggests that the use of Tat as a single component of a vaccine for HIV-1 may not be a viable approach.

### Example 5

### Administration of a Chimeric rev-tat-nef Vaccine to Prevent or Treat HIV Infection

An example of use of the vaccine to treat a patient at risk for HIV-1 infection is provided in this Example. A patient is injected with an attenuated pox virus vector NYVAC carrying a ref-tat-nef chimeric gene designed in accordance with Example 1. The injection comprises about 10⁸ pfU of the pox virus.

The patient's immune responses is evaluated (CD4+ proliferative response; cytotoxic CD8+ T-cell activity, etc.) and a decision is made as to whether and when to immunize again. Typically, a maximum of three to four immunizations with NYVAC-ref-tat-nef is considered. This regimen could be followed by three to four immunizations with ALVAC-ref-tat-nef (carrying a similar HIV-I genetic content). A DNA-only i.e., DNA that is not in a viral vector, vaccine can also be administered, e.g., preceding NYVAC administration.

The vaccine regimen is administered with IL-2, preferably at low doses such as 100,000 to 200,000 units of IL-2 administered daily. CD40⁺ ligand can also be included in the treatment protocol, either by itself or administerd in conjunction with the IL-2 treatment.

The examples provided are by way of illustration only and not limiting. Those of skill will readily recognize a variety of noncritical parameters which could be changed or modified to yield essentially similar results.

1. An expression vector comprising a nucleic acid encoding a chimeric rev, tat, and nef polypeptide.

2. An expression vector of claim 1, wherein the vector is a viral vector.

3. The expression vector of claim 2, wherein the viral vector is an attenuated pox virus vector.

4. The expression vector of claim 3, wherein the attenuated pox virus vector is NYVAC, ALVAC, MVA, or fowlpox.

5. The expression vector of claim 1, wherein the viral vector is an adenovirus vector, an adeno-associated virus vector, or a Venezuelan equine encephalomyelitis virus vector.

6. The expression vector of claim 1, wherein the chimeric polypeptide expressed by the vector comprises functional domains of tat and nef that are disrupted.

7. The expression vector of claim 1, wherein the chimeric polypeptide expressed by the vector lacks a Rev nuclear localization signal, a Rev RNA-binding domain, and a Tat RNA-binding domain.

8. The expression vector of claim 1, wherein the chimeric polypeptide expressed by the vector lacks an N-terminal Nef myristylation signal.

9. The expression vector of claim 1, wherein the chimeric polypeptide expressed by the vector is Retanef.

10. The expression vector of claim 9, further wherein the expression vector is an attenuated pox virus vector that is NYVAC or ALVAC.

11. A method of inducing an immune response comprising administering a first expression vector comprising a nucleic acid sequence encoding a chimeric rev, tat, and nef polypeptide, wherein the expression vector enters the cells of the recipient and intracellularly produce rev, tat, and nef-specific peptides that are presented on the cell's MHC class I molecules in an amount sufficient to stimulate a CD8⁺ response.

12. The method of claim 11, wherein the chimeric rev, tat, and nef polypeptide is retanef.

13. The method of claim 11, wherein the expression vector is a viral vector.

14. The method of claim 13, wherein the viral vector is an attenuated pox virus vector.

15. The method of claim 14, wherein the attenuated pox virus vector is selected from the group consisting of NYVAC and ALVAC.

16. The method of claim 13, wherein the viral vector is an adenovirus vector, an adeno-associated virus vector, or a Venezuelan equine encephalomyelitis virus vector.

17. The method of claim 12, further wherein the chimeric rev, tat, and nef polypeptide is retanef.

18. The method of claim 11, further comprising administering a second expression vector comprising a nucleic acid sequence encoding a chimeric rev, tat, and nef polypeptide, wherein the second expression vector is administered as naked DNA.

19. The method of claim 18, further comprising administering a third expression vector comprising a nucleic acid sequence encoding an HIV structural protein.

20. The method of claim 19, wherein the expression vector encodes HIV gag, pol, and env.

21. The method of claim 18, wherein the chimeric rev, tat and nef polypeptide is retanef.

22. A chimeric rev, tat, and nef polypeptide.

23. The chimeric polypeptide of claim 22, wherein the polypeptide is Retanef.

24. A method of inducing an immune response comprising administering a peptide of claim 22.
